(19) United States
(12) Patent Application Publication
(10) Pub. No.: US 2020/0197667 A1
(43) Pub. Date: Jun. 25, 2020

(54) INTRAVENOUS CATHETER

(71) Applicant: MedSource International LLC, Chaska, MN (US)

(72) Inventor: Neeraj Gupta, Gurgaon (IN)

(21) Appl. No.: 16/642,113

(22) PCT Filed: Mar. 28, 2018
(86) PCT No.: PCT/IN2018/050178

§ 371 Date: Feb. 26, 2020

(30) Foreign Application Priority Data
Feb. 13, 2018 (IN) 201811005453

(51) Int. Cl. A61M 25/06; A61M 25/00

(52) U.S. Cl. CPC A61M 25/0618; A61M 25/0097; A61M 25/0606; A61M 25/0693

(57) ABSTRACT

The intravenous catheter (100, 200) includes the flexible plastic catheter tube (102) having proximal end (104) and distal end (106), the catheter hub (112, 112-A, 112-B, 112-C, 198) having inner chamber (114, 202) influid connection the catheter tube (102). The intravenous catheter (100, 200) further includes the needle (110) adapted to extend along the catheter tube (102). The intravenous catheter further includes the needle cover (146, 146-A) connected to the catheter hub (112, 112-A, 112-B, 112-C, 198). The needle cover (146, 146-A) has the tubular sleeve (150, 150-A) with the slit (152, 152-A) and the bore (154, 154-A) for receiving the needle (110) such that when the needle (110) is retracted from the needle cover (146, 146-A), the needle cover (146, 146-A) which is in tight fit relationship with the catheter hub (112, 112-A, 112-B, 112-C, 198) disengages the catheter hub (112, 112-A, 112-B, 112-C, 198).

### FIELD OF INVENTION

The present disclosure is related to a medical device, more particularly relates to an intravenous catheter protecting individuals from injuries during its usage.

### BACKGROUND OF THE INVENTION

Over-needle catheters have been in existence from many years and now commonly used in hospitals all over the world. Though such catheters have seen many improvements in the past, a simple and reliable device that protects a user from a needle stick injuries is not so yet prevalent. The risk associated with such injuries are very high, because of high prevalence of communicable diseases among patients who reach hospitals and especially in emergency rooms of hospitals where cannulation treatment must be initiated immediately. The cost associated with such needle stick injuries are also very high.

One limitation associated with the existing catheters was found in threaded cap used for closing a secondary or an outer port. The threaded cap may typically be used to close the outer port. However, it may cause an injury due to rotation or twisting of the catheter inserted into the patient's skin, when the cap is rotated either for closing/opening of the outer port. The cap may be required to avoid infections that may be caused due to direct exposure to an outside atmosphere or air. The threaded cap closed at the outer port may often get stuck due to overtightening and thus may require an additional effort/force in order to open the cap. This often may lead to rotation of the catheter inserted into the patient's skin and may cause injuries to veins or skin of the patient.

Another limitation in the existing catheters was found to be associated with invisibility of the blood flow in the catheters after puncturing of the vein of the patient. When the catheter is inserted along with the needle to puncture the vein, the blood from patient's vein flows through the catheter and reaches to a flashback chamber located at a rear end or proximal end of the catheter. As the operator's hand is covered over the catheter during the process, the flashback chamber coming under the operator's hand does not provide visibility of the blood flow and the operator may need to turn-over or lift the hand to uncover the portion of the catheter to view the flashback chamber to ascertain the blood flow. This may cause discomfort to the patient and also may cause needle injuries to the patient's vein/skin.

The Indian Patent application number 3031/DEL/2014, (hereafter referred to as the Pat '3031) provides a catheter device. One of the problem associated with the pat '3031 is that when the needle is retracted from the needle cover, the needle cover does not disengage from the catheter hub and it was experienced by the operators that more force is required to disengage the needle cover from the catheter hub. Another problem associated with the needle disclosed in the pat '3031 is that, the needle does not engage the needle cover with the catheter hub affecting the functionality of the catheter. This may lead to manipulation of the catheter device by the operator to extract the needle cover from the hub, which in turn may disturb the vein and may cause pain to the patients and may cause damage to the veins.

Therefore, the present disclosure is directed to overcome one or more of the problems as set forth above.

### SUMMARY OF THE INVENTION

The present disclosure provides for an intravenous catheter. The catheter includes a flexible plastic catheter tube having a proximal end and a distal end. The catheter further includes catheter hub having an inner chamber in fluid connection with the catheter tube forming a first fluid pathway. The catheter hub having a distal end is connected to the proximal end of the catheter tube having a bore. The catheter further includes a needle having a proximal end, a tip at a distal end and an enlarged dimension feature towards the distal end. The needle is adapted to extend along a length of the bore of the catheter tube and the proximal end of the needle is press fitted in a needle hub. The needle hub is connected to a flashback chamber through an extended portion of the needle hub. The catheter further includes a needle cover connected to the catheter hub by at least one projection and at least one recess configuration. The needle cover of the catheter has a tubular sleeve with an axially oriented radially inwards extending slit and a bore for receiving the needle such that when the needle is retracted from the needle cover, the needle cover which is in tight fit relationship with the catheter hub disengages the catheter hub.

In an embodiment of the present disclosure, the tubular sleeve includes the recess on its outer surface configured to engage with the projection in the catheter hub, the recess is a groove or cavity on the outer surface of the tubular sleeve.

In an embodiment of the present disclosure, the tubular sleeve includes a projection on the outer surface of the tubular sleeve configured to engage with a recess in the catheter hub, the recess is a groove or cavity on an inner surface of the catheter hub.

In an embodiment of the present disclosure, the needle cover having the tubular sleeve with the slit and the bore is adapted to receive the needle such that when the needle is inserted into the needle cover, the extended dimension feature of the needle pushes the slit outwardly for locking of the needle cover with the catheter hub by engagement of the projection with the recess.

In an embodiment of the present disclosure, the projection is radially extending ring like structure or a protruding feature designed to mesh with the recess.

In an embodiment of the present disclosure, the catheter hub includes an outer port abutting on an outer surface forming a second fluid pathway, the second fluid pathway is in fluid communication with the first fluid pathway.

In an embodiment of the present disclosure, the outer port is provided with a dispensing cap in which a hinge of the dispensing cap has a spring action.

In an embodiment of the present disclosure, the dispensing cap and an outer port are connected together by the hinge urging the dispensing cap to freely swing to a fully open or a fully closed position when pushed in respective directions.

In an embodiment of the present disclosure, the catheter further includes an elongated tube connected to the extended portion of the needle hub.

In an embodiment of the present disclosure, the elongated tube includes an inner chamber with a first end and a second end, such that the second end is configured to be connected to the extended portion of the needle hub.

In an embodiment of the present disclosure, the elongated tube includes a hydrophobic filter disposed within the inner chamber proximal to the first end.

In an embodiment of the present disclosure, the catheter further includes a rigid conical guide member abutting a disc in the catheter hub, the disc is made of silicone.

In an embodiment of the present disclosure, the needle cover is connected to a casing, the casing being detachably connected to the needle cover via one or more ribs and to the needle hub via one or more hooks inserted into one or more slots of the needle hub.

In an embodiment of the present disclosure, the needle is a hypodermic needle having a bevelled tip.

Other features and aspects of this disclosure will be apparent from the following description and the accompanying drawings.

### BRIEF DESCRIPTION OF ACCOMPANYING DRAWINGS

FIG. 1 shows a perspective view of an intravenous catheter, according to an embodiment of the present disclosure;

FIG. 2 shows a sectional view of the intravenous catheter along axis A-A′ in FIG. 1, according to an embodiment of the present disclosure;

FIG. 3 shows a perspective view of an assembly of the catheter hub of the intravenous catheter with the needle cover of the intravenous catheter shown in FIGS. 1 and 2, according to an embodiment of the present disclosure;

FIG. 4 shows a sectional view of the assembly along axis B-B′ shown in FIG. 3, according to an embodiment of the present disclosure;

FIG. 5 shows a perspective view of an assembly of a catheter hub of the intravenous catheter with the needle cover of the intravenous catheter, according to another embodiment of the present disclosure;

FIG. 6 shows a sectional view of the assembly along axis C-C′ shown in FIG. 5, according to another embodiment of the present disclosure;

FIG. 7 shows a perspective view of an assembly of a catheter hub of the intravenous catheter with a needle cover of the intravenous catheter, according to another embodiment of the present disclosure;

FIG. 8 shows a sectional view of the assembly along axis D-D′ shown in FIG. 7, according to another embodiment of the present disclosure;

FIG. 9 shows an enlarged view of potions “E1”, “E2”, “E3” indicated in FIGS. 4, 6 and 8, according to one or more embodiments of the present disclosure;

FIG. 10 shows a perspective view of the catheter hub shown in FIGS. 1 and 2, according to an embodiment of the present disclosure;

FIG. 11 shows a sectional view of the catheter hub along axis F-F′ shown in FIG. 10, according to an embodiment of the present disclosure;

FIG. 12 shows a perspective view of an inner profile of a catheter hub having one or more protruded profiles, according to an embodiment of the present disclosure;

FIG. 13 shows a side view of an assembly having the catheter hub shown in FIG. 12 and the needle covers shown in FIGS. 1-8, according to one or more embodiment of the present disclosure;

FIG. 14 shows a perspective view of the needle cover shown in FIGS. 1-8, according to one or more embodiments of the present disclosure;

FIG. 15 shows a side view of the needle cover shown in FIG. 14, according to one or more embodiments of the present disclosure;

FIG. 16 shows a side view of an assembly of a catheter hub with a needle cover, according to another embodiment of the present disclosure;

FIG. 17 shows a side view of a needle cover shown in FIG. 16, according to another embodiment of the present invention;

FIG. 18 shows a portion “G” from FIG. 16, according to another embodiment of the present invention;

FIG. 19 shows a perspective view of a portion of an inner profile of the catheter hub shown in FIGS.16-18, according to another embodiment of the present invention;

FIG. 20 shows a plan view of an intravenous catheter, according to another embodiment of the present disclosure;

FIG. 21 shows a plan view of a catheter hub shown in FIG. 20, according to an embodiment of the present disclosure;

FIG. 22 shows a plan view of an elongated tube shown in FIG. 20, according to an embodiment of the present disclosure;

FIG. 23 shows an exploded view of a holder cover with a needle hub, of the intravenous catheters shown in FIGS. 1, 2 and 20, according to one or more embodiment of the present disclosure;

FIG. 24A shows a perspective view of a cap assembly of intravenous catheter shown in FIGS. 1 and 2, in closed position, according to an embodiment of the present disclosure;

FIG. 24B shows a perspective view of a cap assembly of intravenous catheter shown in FIGS. 1 and 2, in open position, according to an embodiment of the present disclosure;

FIG. 25A shows a perspective view of a cap assembly for intravenous catheter shown in FIGS. 1 and 2, in closed position, according to another embodiment of the present disclosure;

FIG. 25B shows a perspective view of a cap assembly for intravenous catheter shown in FIGS. 1 and 2, in open position, according to another embodiment of the present disclosure; and

FIGS. 26A and 26B shows various views of a needle, according to another embodiment of the present disclosure.

### DETAILED DESCRIPTION OF THE INVENTION

Provided below is a non-limiting exemplary embodiment of the present disclosure and a reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claim.

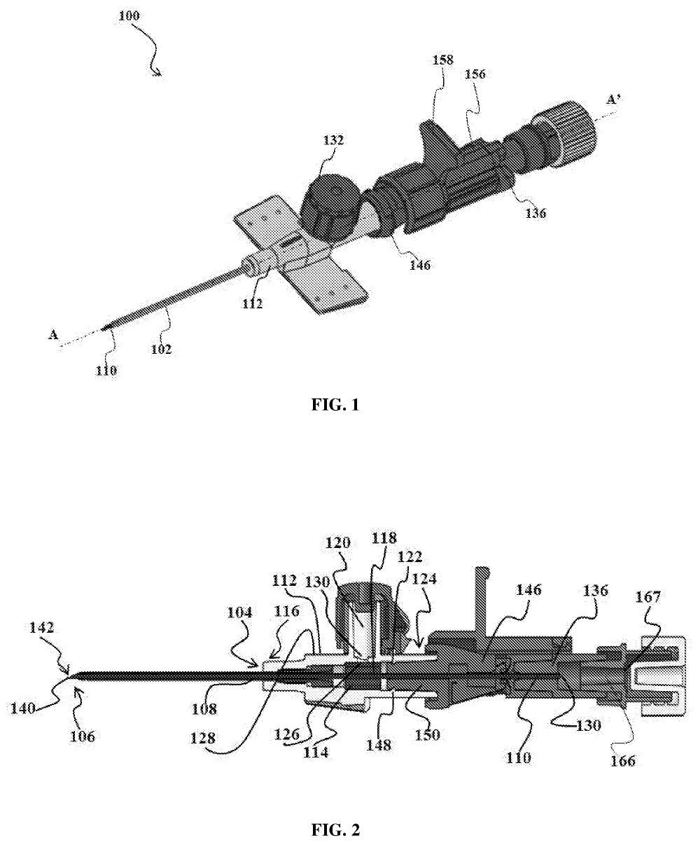

Referring to FIGS. 1 and 2 illustrates a perspective view and a sectional view of a catheter (100), according to an embodiment of the present disclosure. The catheter (100) is a medical device used onto patients undergoing a treatment for administration of a medication fluid or gases by an intravenous therapy or removal of body fluid for example ‘blood sample’, from the patients for a laboratory analysis or the catheter may be used to carry-out other tasks based on a type of catheter. In the illustrated figures, the catheter (100) includes, but not limited to, an intravenous catheter (100). The term ‘intravenous catheter (100)’ as referred herein in the present disclosure will be interchangeably used by a term ‘catheter (100)’. It is to be understood that both the terms ‘intravenous catheter (100)’ and ‘catheter (100)’ relates to a same device. The intravenous catheter (100) as illustrated in the FIG. 1 includes a catheter tube (102).

The catheter tube (102) includes a proximal end (104) and a distal end (106) opposite to the proximal end (104). It is to be understood that the term ‘proximal end’ as used in the present disclosure may be defined as an end closer to an operator (not shown) who operates the catheter (100). The term ‘distal end’ as used in the present disclosure may be defined as an end opposite to the ‘proximal end’ and away from the operator. The catheter tube (102) can be made of a flexible or soft material, including, but not limited to, a plastic or a polymer or a bio-compatible material which is flexible/soft in nature. A stiffness of the catheter tube (102) may be varied based on an application of the catheter tube (102), by changing the material of the catheter tube (102). The catheter tube (102) is a thin elongated tubular structure having a bore (108). The bore (108) is adapted to encase a needle (110) of the intravenous catheter (100). The proximal end (104) of the catheter tube (102) can be adapted to fix with a catheter hub (112) of the intravenous catheter (100) by a method including, but not limited to, a press fitting or adhesive bonding or any other methods known in the art to serve a purpose of holding the catheter tube (102) with the catheter hub (112). In an embodiment, the catheter hub (112) can be made of a bio-compatible material which can be rigid and can securely hold components associated with it.

The catheter hub (112) of the intravenous catheter (100), includes an inner chamber (114) (more clearly shown in FIGS. 4 and 11) adapted to be in a fluidic connection with the catheter tube (102). The catheter hub (112) further includes a distal end (116) connected to the proximal end (104) of the catheter tube (102) forming a first fluid pathway (118) between the catheter tube (102) and the catheter hub (112). The inner chamber (114) of the catheter hub (112) may be configured into a cylindrical passage (120) for allowing the needle (110) to pass through the catheter hub (112) and pass through along a length of the bore (108) of the elongated tubular structure of the catheter tube (102). The inner chamber (114) of the catheter hub (112) further includes at least one projection (122) oriented in a radially inward manner and at a proximal end (124) of the catheter hub (112) according to an exemplary embodiment of the present disclosure.

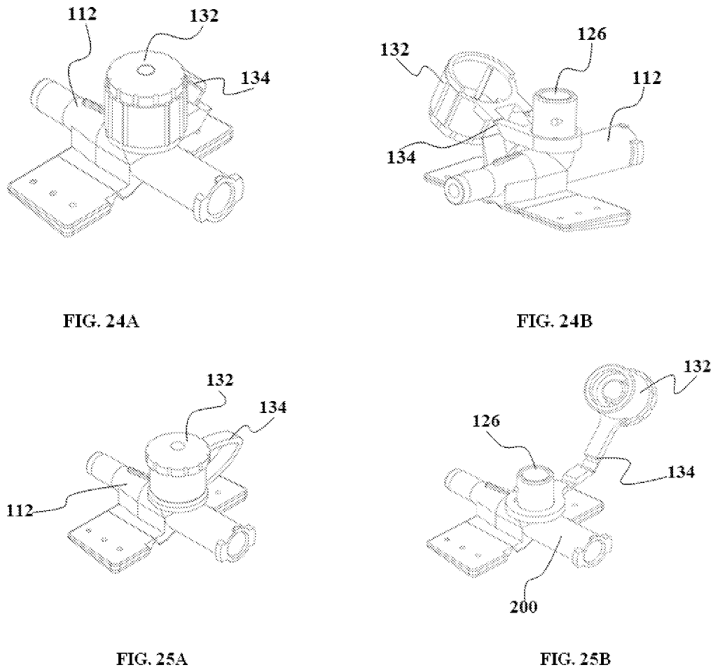

The catheter hub (112) further includes an outer port (126) abutting on an outer surface (128) of the catheter hub (112) forming a second fluid pathway (130) in communication with the cylindrical passage (120) of the catheter hub (112). Therefore, the second fluid pathway (130) is made to be in fluid communication with the first fluid pathway (118) of the catheter hub (112). Thus, the intravenous catheter (100) in this embodiment is adapted to be having a two-way fluid mechanism. The outer port (126) of the catheter hub (112) may be provided with a dispensing cap (132) for closing and opening of the outer port (126). The dispensing cap (132) may be operated to open and close via a hinge (134) (more clearly shown in FIGS. 24A, 24B, 25A and 25B) or any other mechanism known in the art. The hinge (134) provides a spring action for closing and opening of the outer port (126) and thus urges the dispensing cap (132) to freely swing to a fully open or to a fully closed position when pushed during respective operations.

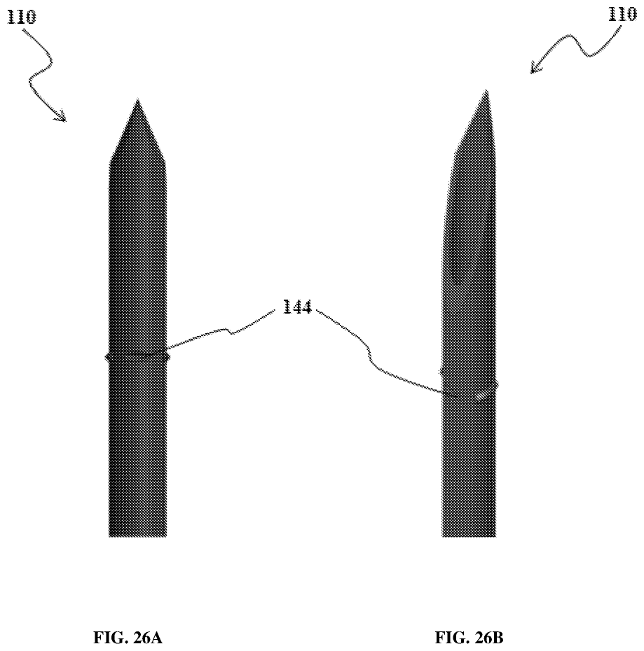

The intravenous catheter (100) further includes a needle hub (136) having the needle (110). The needle (110) in the illustrated embodiment is a needle is a hypodermic needle having a bevelled tip. The needle (110) includes a proximal end (138), a tip (140) at a distal end (142) and an enlarged dimension feature (144) (shown in FIGS. 26A and 26B) towards the distal end (142) of the needle (110). The enlarged dimension feature (144) restricts movement of the needle (110) in a needle cover (146). The needle cover (146) is connected to the catheter hub (112) in such a way that a recess (148) (shown in FIGS. 2 and 4) on the needle cover (146) is engaged with the projection (122) (shown in FIGS. 2 and 4) of the catheter hub (112). The proximal end (138) of the needle (110) is connected to the needle hub (136) to hold the needle (110) firmly. The needle (110) may be fixed to the needle hub (136) through a process including, but not limited to, a press fitting, an adhesive, or any other process known in the art. The distal end (142) of the needle (110) is made to pass through the needle cover (146), the catheter hub (112) and the catheter tube (102).

As shown in FIG. 2 the needle cover (146) is adapted to be slidably disposed into the needle (110) connected to the needle hub (136). That is to say, the needle cover (146) is disposed in between the catheter hub (112) and the needle hub (136), and the distal end (142) of the needle (110) is inserted through the catheter hub (112) and the needle cover (146), such that, the needle cover (146) partially encases the needle (110) and is connected to the catheter hub (112). In an embodiment, the needle cover (146) is connected to the catheter hub (112) by the projection (122) and the recess (148) configuration.

The needle cover (146) further includes a tubular sleeve (150) with an axially oriented radially inwards extending slit (152) and a bore (154) (more clearly shown in FIG. 4) for receiving the needle (110), such that when the needle (110) is withdrawn from the catheter hub (112), the needle cover (146) which is in tight fit relationship with the catheter hub (112) disengages the catheter hub (112). The catheter (100) further includes a casing (156) which encases both the needle cover (146) and the needle hub (136) in a partial manner. The casing (156) may be provided with a thumb grip (158) for providing a gripping to the operator during insertion or retraction of the catheter (100).

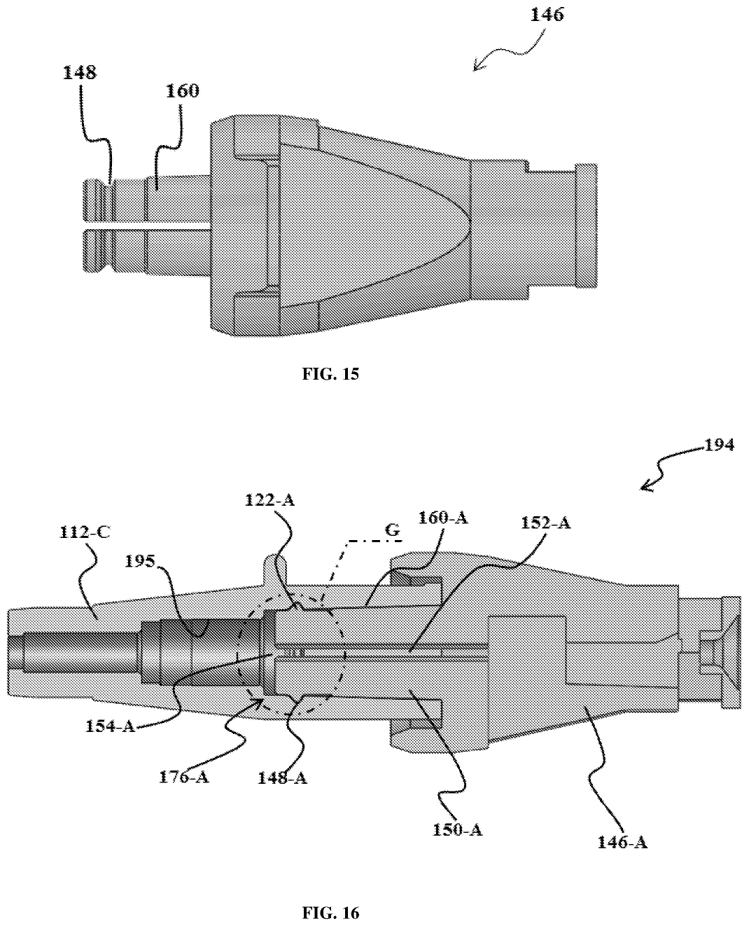

The tubular sleeve (150) having an outer surface (160) includes the recess (148) (more clearly shown in FIGS. 14 and 15). The tubular sleeve (150) is detachably connected to the catheter hub (112) and engaged with the projection (122) on the catheter hub (112), such that when the needle (110) is withdrawn, the needle cover (146) which is in tight fit relationship with the catheter hub (112) disengages the catheter hub (112) from the tubular sleeve (150) through the enlarged dimension feature (144) of the needle (110).

The needle (110) having the enlarged dimension feature (144) makes a diameter of the needle (110) slightly higher than a diameter of the bore (154) of the tubular sleeve (150). Thus, the slit (152) and the bore (154) of the tubular sleeve (150) helps in ensuring a locking and unlocking of the needle cover (146) with and from the catheter hub (112), respectively. That is to say, when the needle (110) is inserted the slit (152) of the tubular sleeve (150), the needle (110) having the enlarged diameter feature (144) pushes the slit (152) of the tubular sleeve (150) radially so that the projection (122) of the catheter hub (112) is made to lock inside the recess (148) of the tubular sleeve (150) for making a locking engagement between the catheter hub (112) and the needle cover (146). The locking engagement between the needle cover (146) and the catheter hub (112) will need to overcome a locking force ranging from about 1 N-1.5 N, to keep an assembly of the needle cover (146) and the catheter hub (112) securely without any mutual disengagement until the catheter (100) is served with a required purpose of puncturing a vein of the patient. Therefore, a normal force required for overcoming the locking force of 1 N- 1.5 N for disengaging the tubular sleeve (150) from the catheter hub (112) may be in a range of 18N-20 N.

When the operator operates to withdraw the needle (110) after puncturing the vein of the patient, the locking engagement between the catheter hub (112) and the needle cover (146) needs to be disengaged to remove the needle (110) from the catheter hub (112). When the needle cover (146) is pulled to withdraw, the needle (110) is withdrawn from the locked engagement of the projection (122) and the recess (148). Thus, the locking force applied between the catheter hub (112) and the needle cover (146) is drastically reduced, and the needle cover (146) can be disengaged from the catheter hub (112) with a force less than 0.5 N, allowing for easy disengagement of the needle cover (146) from the catheter hub (112).

Further, the needle (110) withdrawn from the vein of the patient gets arrested within the needle cover (146). Thus, the needle (110) which is withdrawn with normal force and being arrested with the needle cover (146), prevents injuries to the operator as the needle (110) does not come in contact with the operator.

The catheter (100) further including the casing (156) receives the needle cover (146) such that the needle cover (146) cannot be manipulated during insertion of the catheter (100) in the vein or before withdrawal of the needle (110) is performed. In an embodiment, the casing (156) is detachably connected to the needle cover (146) via ribs (not shown). Also, the casing (156) is detachably connected to the needle hub (136) via one or more hooks (162) inserted into one or more slots (164) provided on the needle hub (136) (as shown in FIG. 23). Further, the needle hub (136) is connected to a flashback chamber (166) through an extended portion (168) of the needle hub (136) and may be closed using a threaded cap (170). The blood/blood sample in the flashback chamber (166) confirms puncturing of the vein by the needle (110). The flashback chamber (166) can additional include a hydrophobic filter (167) for preventing spillage of the blood/blood sample from the flash back chamber (166).

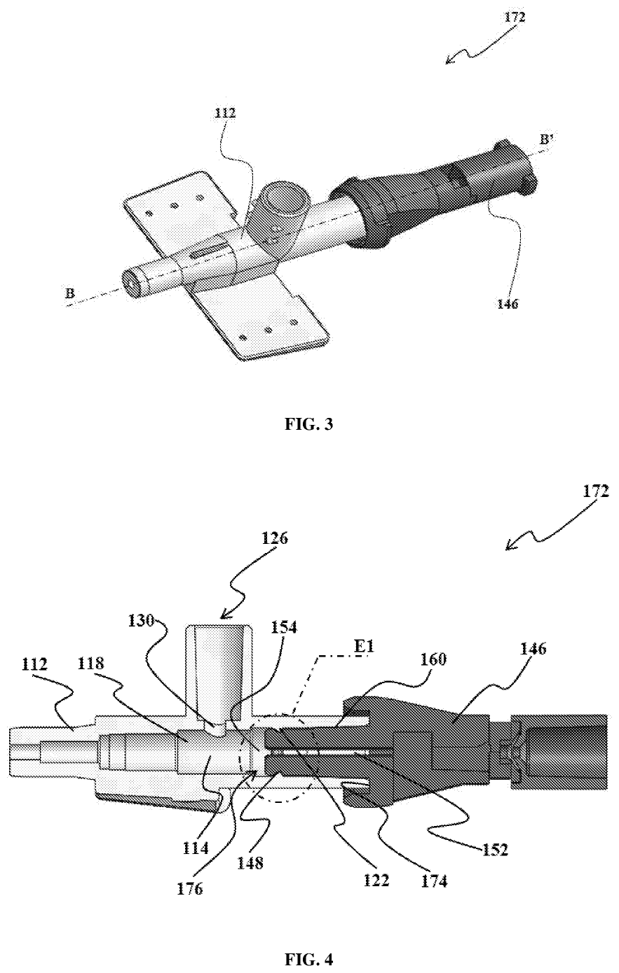

Referring to FIGS. 3 and 4, which illustrates a perspective view and a sectional view along axis B-B′ of an assembly (172) of the catheter hub (112) of the intravenous catheter (100) with the needle cover (146) of the intravenous catheter (100) shown in FIGS. 1 and 2, according to an embodiment of the present disclosure. The catheter hub (112) in this embodiment includes the outer port (126) of the second fluid pathway (130) which is in communication with the first fluid pathway (118) of the catheter hub (112).

The needle cover (146) in the illustrated figure includes the tubular sleeve (150) extending axially from an inner portion (174) of the needle cover (146). The tubular sleeve (150) includes the bore (154) adapted to allow the needle (110) to pass through. Further, the tubular sleeve (150) includes the slit (152) configured axially. The tubular sleeve (150) further includes the recess (148) configured radially on the outer surface (160) of the tubular sleeve (150) and is disposed at a distal end (176) of the tubular sleeve (150). The needle cover (146) and the catheter hub (112) are connected to each other through the recess (148) and the projection (122) of the catheter hub (112). It may be understood that the tubular sleeve (150) may be configured with more than one recesses and the catheter hub (112) may also be provided with more than one projections which may be based on the requirement.

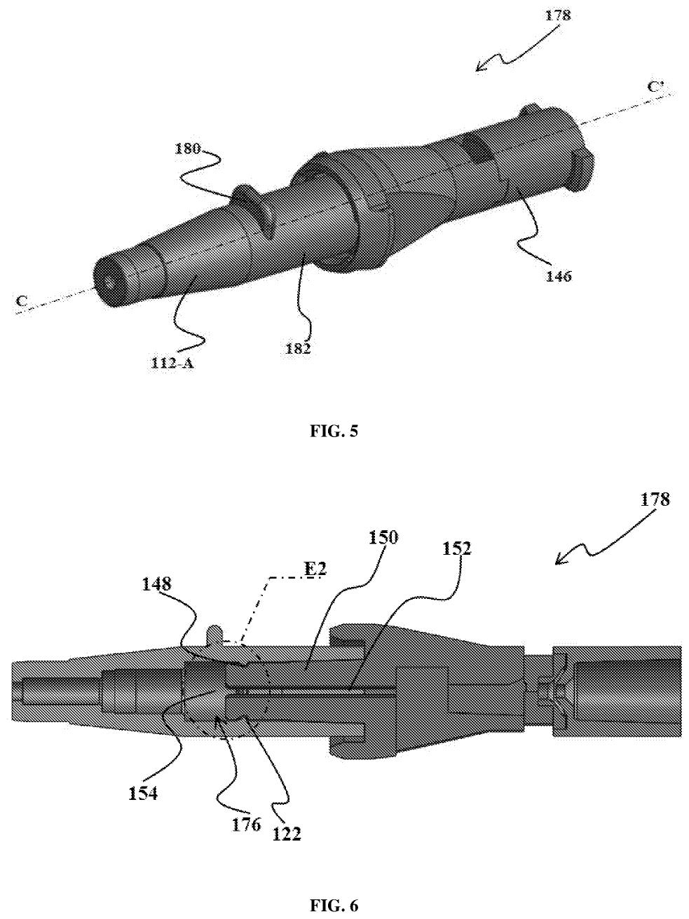

Referring to FIGS. 5 and 6 illustrates a perspective view and sectional view along axis C-C′ of an assembly (178) of a catheter hub (112-A) of the intravenous catheter (100) with the needle cover (146) of the intravenous catheter (100), according to another embodiment of the present disclosure. The catheter hub (112-A) in this embodiment includes a thumb portion (180) at an outer surface (182) of the catheter hub (112-A).

The needle cover (146) in the illustrated figure includes the tubular sleeve (150) extending axially from an inner portion (174) of the needle cover (146). The tubular sleeve (150) includes the bore (154) adapted to allow the needle (110) to pass through. Further, the tubular sleeve (150) includes the slit (152) configured axially. The tubular sleeve further includes the recess (148) configured radially on the outer surface (160) of the tubular sleeve (150) and is disposed at the distal end (176) of the tubular sleeve (150). The needle cover (146) and the catheter hub (112-A) are connected to each other through the recess (148) and the projection (122) of the catheter hub (112-A). It may be understood that the tubular sleeve (150) may be configured with more than one recesses and the catheter hub (112-A) may also be provided with more than one projections which may be based on the requirement.

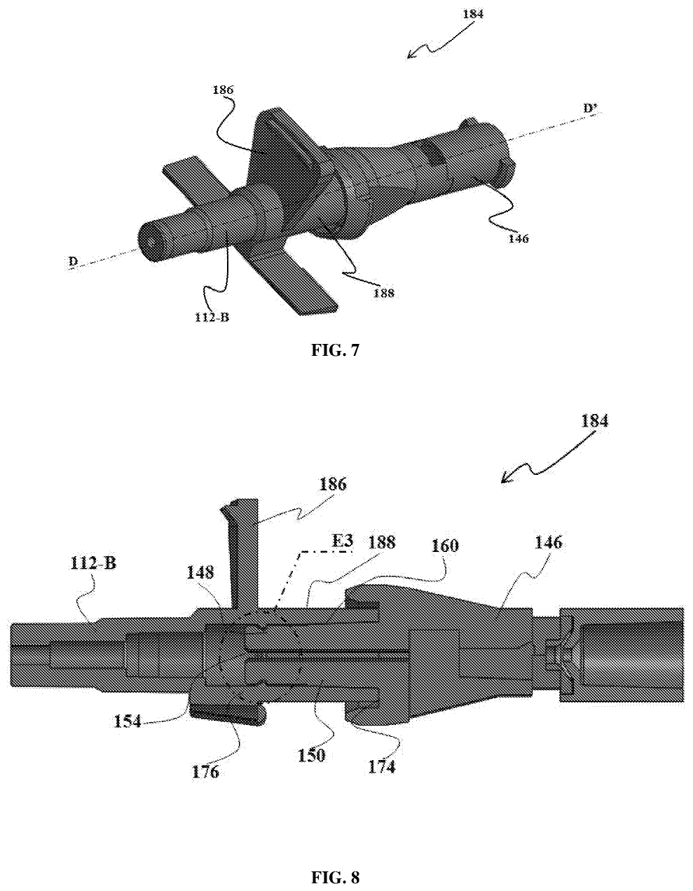

Referring to FIGS. 7 and 8 illustrates a perspective view and sectional view along axis D-D′ of an assembly (184) of a catheter hub (112-B) of the intravenous catheter (100) with the needle cover (146) of the intravenous catheter (100), according to another embodiment of the present disclosure. The catheter hub (112-B) in this embodiment includes another thumb portion (186) at an outer surface (188) of the catheter hub (112-B).

The needle cover (146) in the illustrated figure includes the tubular sleeve (150) extending axially from THE inner portion (174) of the needle cover (146). The tubular sleeve (150) includes the bore (154) adapted to allow the needle (110) to pass through. Further, the tubular sleeve (150) includes the slit (152) configured axially. The tubular sleeve (150) further includes the recess (148) configured radially on the outer surface (160) of the tubular sleeve (150) and is disposed at the distal end (176) of the tubular sleeve (150). The needle cover (146) and the catheter hub (112-B) are connected to each other through the recess (148) and the projection (122) of the catheter hub (112-B). It may be understood that the tubular sleeve (150) may be configured with more than one recesses and the catheter hub (112-B) may also be provided with more than one projections which may be based on the requirement.

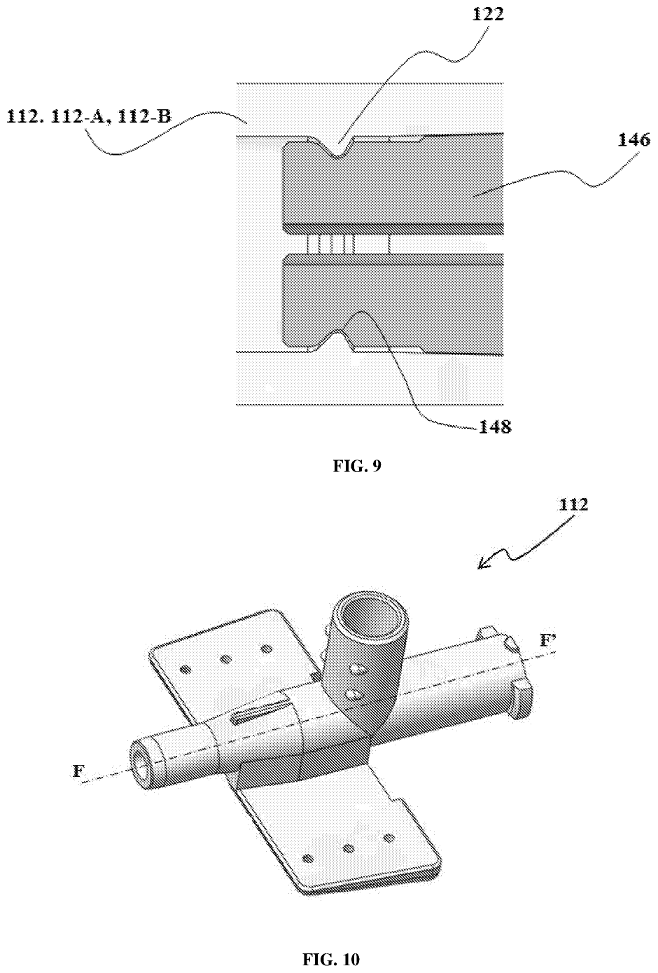

FIG. 9 illustrates a magnified view of portions “E1”, “E2”, “E3” indicated in FIGS. 4, 6, and 8, according to one or more embodiments of the present disclosure. The magnified view of portions “E1”, “E2”, “E3” illustrates the locking engagement of the projection (122) of the catheter hub (112, 112-A, 112-B) with the recess (148) of the needle cover (146) when the needle (110) is inserted into the bore (154) of the tubular sleeve (150) of the needle cover (146).

Referring to FIGS. 10 and 11, which illustrates a perspective view and a sectional view along axis F-F′ in FIG. 10, of the catheter hub (112) shown in FIGS. 1 and 2, according to an embodiment of the present disclosure. The catheter hub (112) having the inner chamber (114) includes the projection (122), radially directing towards the inner chamber (114) (as shown in FIG. 11). In the illustrated embodiment, the inner chamber (114) of the catheter hub (112) includes a single projection; however, it is to be understood that the inner chamber (114) of the catheter hub (112) may be provided with more than one projections for providing the locking engagement between the catheter hub (112) and the needle cover (146).

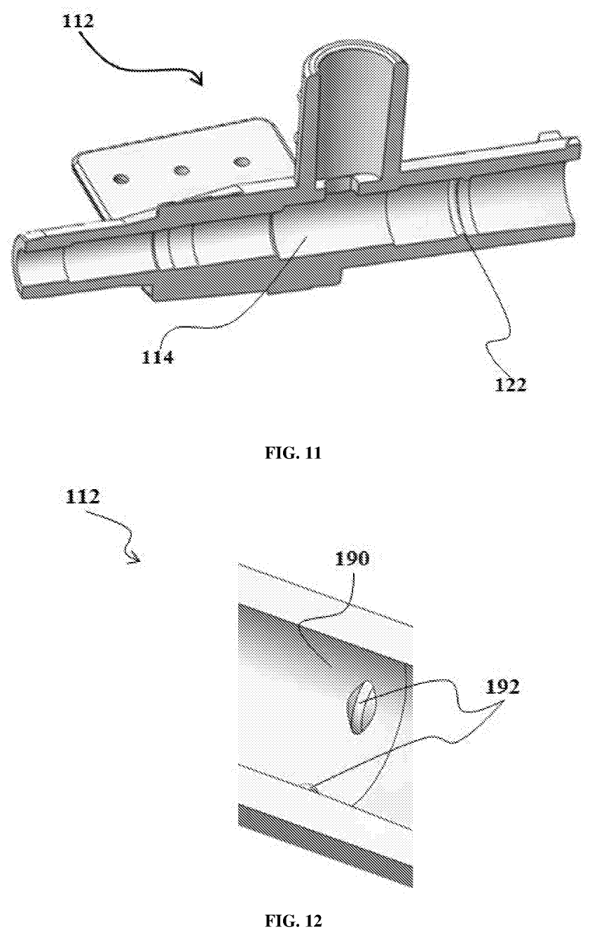

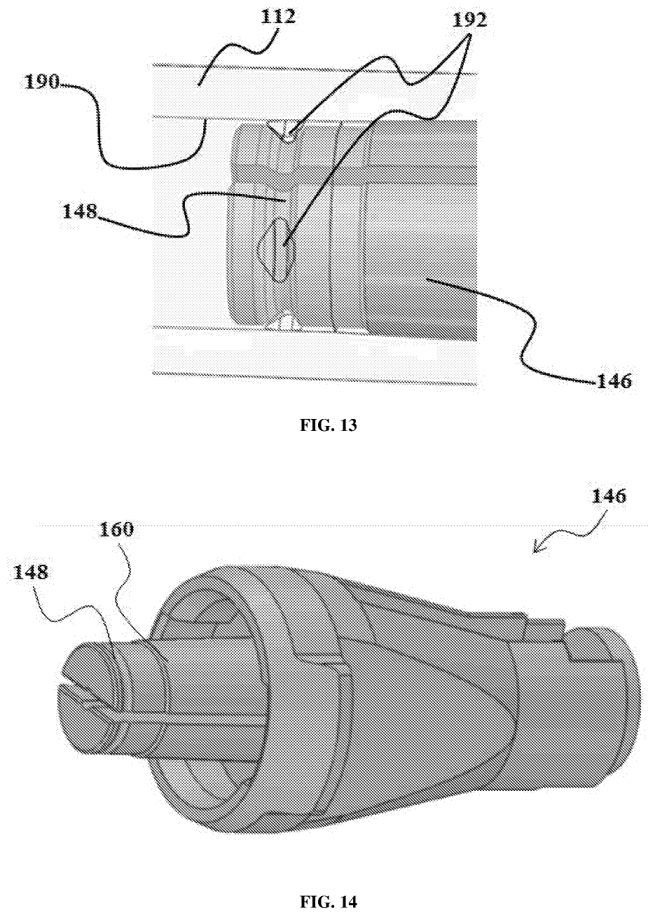

Referring to FIGS. 12 and 13, which illustrates an inner surface (190) of the catheter hub (112) according to another embodiment of the present disclosure. The inner surface (190) of the catheter hub (112) may include projections in an intermittent manner. That is to say, the inner chamber (190) may be provided with one or more dotted projections (192) which are radially directed towards the inner chamber (190). The dotted projections (192) in this embodiment may include same profile as that of the projection (122) of the catheter hub (112) shown in FIG. 11. In the illustrated embodiment, the inner chamber (190) of the catheter hub (112) includes a four dotted projections spaced at equally around the inner surface (190) of the catheter hub (112). However, it is to be understood that the inner surface (190) of the catheter hub may be configured with more than four dotted projections for locking the catheter hub (112) with the needle cover (146) having the recess (148) matching with a profile of the dotted projections (192).

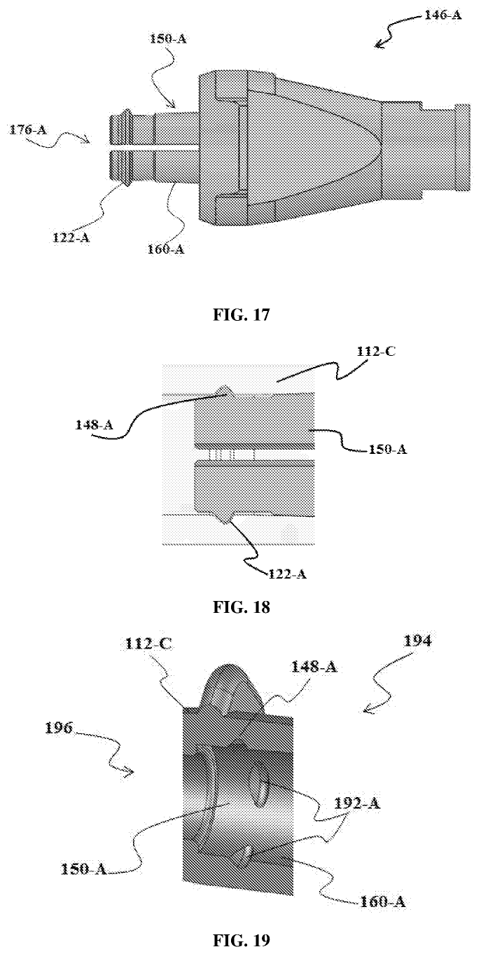

FIG. 16 illustrates a side view of an assembly (194) of a catheter hub (112-C) with a needle cover (146-A), according to another embodiment of the present disclosure. In the illustrated embodiment, the catheter hub (112-C) includes at least one annular recess (148-A) on an inner surface (195) of the catheter hub (112-C). The needle cover (146-A) in the illustrated figure includes a tubular sleeve (150-A) extending axially from an inside portion (174-A) of the needle cover (146-A). The tubular sleeve (150-A) includes a bore (154-A) adapted to allow the needle (110) to pass through. Further, the tubular sleeve (150-A) includes a slit (152-A) configured axially. The tubular sleeve (150-A) further includes at least one projection (122-A) configured radially on an outer surface (160-A) of the tubular sleeve (150-A) and is disposed proximal to at a distal end (176-A) of the tubular sleeve (150-A) (more clearly shown in FIG. 17). The needle cover (146-A) and the catheter hub (112-C) are connected to each other through the at least one projection (122-A) of the tubular sleeve (150-A) of the needle cover (146-A) and the at least one recess (148-A) of the catheter hub (112-C) (more clearly shown in portion “G” in FIG. 18)

FIG. 19 illustrates a cut-away view of a portion of an inner profile (196) of the assembly (194) of catheter hub (112-C) shown in FIGS. 16-18 and the needle cover (146-A), according to another embodiment of the present invention. In the illustrated figure, the outer surface (160-A) of the needle cover (146-A) includes projection in an intermittent manner in an alternate embodiment of the needle cover (146-A). That is to say, the outer surface (160-A) of the tubular sleeve (150-A) may be provided with one or more dotted projections (192-A) which are radially directing towards the inner annular recess (148-A) of the catheter hub (112-C). In the illustrated embodiment, the outer surface (160-A) of the needle cover (146-A) includes a four dotted projections spaced at equally. However, it is to be understood that the outer surface (160-A) of the needle cover (146-A) may be configured with more than four dotted projections for locking the needle cover (146-A) with the catheter hub (112-C).

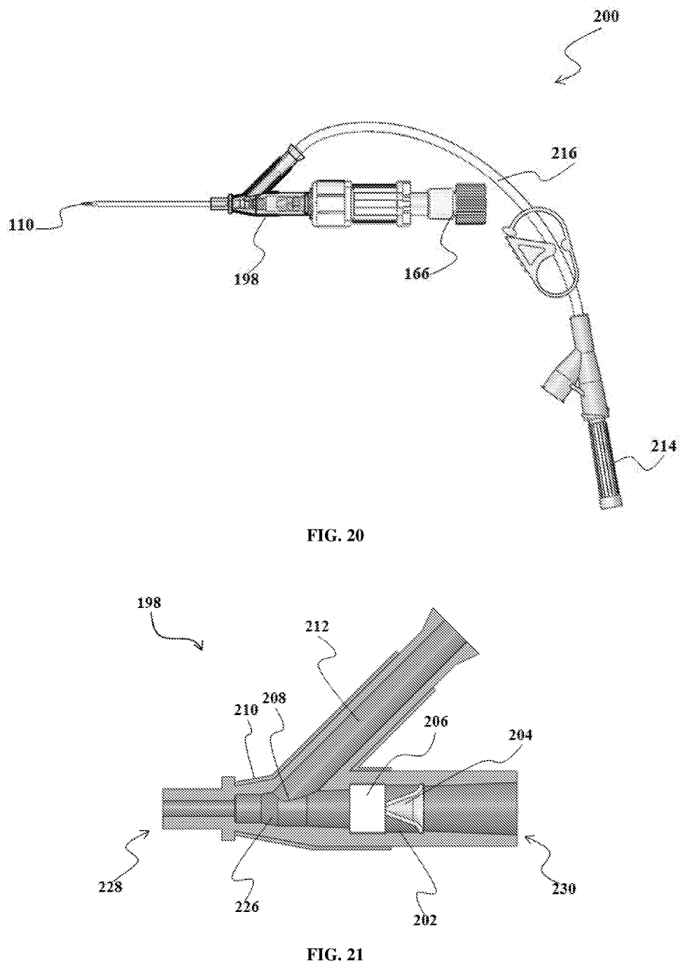

Referring to FIGS. 20 and 21 which illustrates a plan view of an intravenous catheter (200) and a catheter hub (198) incorporated into the intravenous catheter (200), according to another embodiment of the present disclosure. The catheter hub (198) illustrates a side view, including an inner chamber (202). The inner chamber (202) accommodates a rigid conical guide member (204) abutting a disc (206). The disc (206) can be made of a material for example a polymer like silicone having a self-sealing property. The self-sealing property of the disc (206) provides sealing when the needle (110) is pierced through the disc (206) and when the needle (110) is withdrawn from the disc (206), a hole (not shown) that is created due to the needle (110) gets closed due to the selfsealing property of the disc (206). Thus, the disc (206) prevents spilling of fluid or blood after retracting the needle (110) from the catheter hub (198) and the rigid conical guide member (204) provides alignment of the needle (110) within an axis of the catheter hub (198). The rigid conical guide member (204) may be made of a plastic material.

The catheter hub (198) further includes an outer port (208) abutting onto an outer surface (210) of the catheter hub (198) forming a second fluid pathway (212). The second fluid pathway (212) is connected to a first fluid pathway (226) of the catheter hub (198). The outer port (208) may be configured to connect with an elongated tube (214) through a flexible tube (216) at a distal end (228) in an example. In another example, the distal end (228) of the elongated tube (214) can be connected to the extended portion (168) of the needle hub (136) when the catheter (100, 200) is required to be fitted with the elongated tube (214) for better visibility of the blood flow.

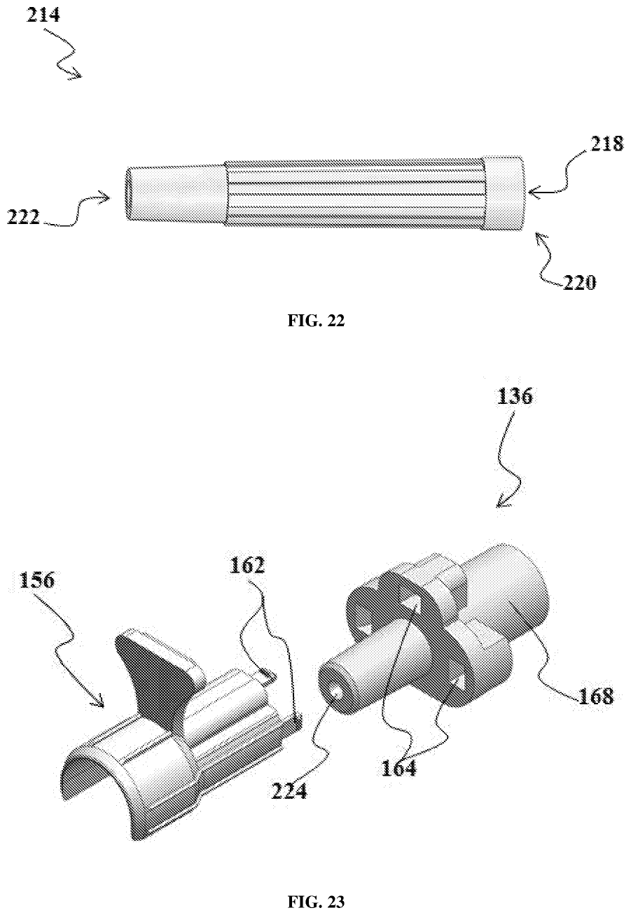

FIG. 22 illustrates a plan view of the elongated tube (214) shown in FIG. 20, according to an embodiment of the present disclosure. The elongated tube (214) in the illustrated embodiment may be provided in place of a flash back chamber (166) as illustrated in the FIGS. 1, 2 and 20. The elongated tube (214) has an inner chamber (218) with a first end (220) and a second end (222), such that the second end (222) is configured to be connected to the extended portion (168) of the needle hub (136) (shown in FIG. 23). The elongated tube (214) being made as a translucent element provides a clear visibility of a passage in the elongated tube (214) which in turn provides a visibility of the blood flow after puncturing of the patient's vein. In an embodiment, the elongated tube (214) may include a hydrophobic filter (not shown) disposed within the inner chamber (218) and located proximal to the first end (220). The hydrophobic filter has a pores which stops the passage of blood from the elongated tube (214).

FIG. 23 illustrates an exploded view of the casing (156) with the needle hub (136), of the intravenous catheters (100, 200) shown in FIGS. 1, 2 and 20, according to one or more embodiment of the present disclosure. The needle hub (136) of the catheter (100, 200) includes a through hole (224) in which the needle (110) is fitted through a method including but not limited to, press fitting or adhesive fixing. The needle hub (136) includes the slots (164) and the extended portion (168) which can be connected to the flash back chamber (166). The needle hub (136) is connected to the casing (156) through the hooks (162), like snap lock member.

### Advantages of the Invention

In an embodiment, the disclosed catheter securely prevents the tip of the needle to come in contact with the user after withdrawing the needle from the needle cover. The needle gets arrested within the needle cover and the tip of the needle rests within the needle cover when the needle is disengaged from the catheter hub. That is to say, the intravenous catheter provides a safety mechanism such that the tip of the needle does not comes in contact with the operator or the patient when the needle is withdraw after piercing the vein of the patient. Thus, serious injuries or infections that would have caused by the needle is prevented.

In an embodiment, the disclosed catheter including the projection and recess locking between the catheter hub and the needle cover holds the catheter hub and the needle cover securely without any loose connection or contact and prevents the unintentional withdrawal of the needle.

In an embodiment, the disclosed catheter including the projection and recess locking between the catheter hub and the needle cover, and the needle having an enlarged dimension feature enables the easy removal of the needle by unlocking the projection from the recess.

In an embodiment, the catheter having the dispensing cap with the hinge provides spring action to the dispensing cap and thereby provides an easy opening and closing of the outer port. Thus, injuries that may be caused due to rotation of the dispensing cap as provided in the existing art are prevented.

In an embodiment, the dispensing cap closes the outer port which prevents infections that may be caused due to exposure of the outer port to the air or external environment as the hinge of the dispensing cap provides the spring action and keeps the outer port closed when not in use.

In an embodiment, the catheter provided with the elongated tube connected to the extended portion of the needle hub provides visibility of the blood flow and thus ascertain the user about piercing or puncturing of the patient's view. Thus, the injuries and discomfort to view the blood flow is avoided.

In an embodiment, as the elongated tube has the hydrophobic filter disposed within the inner chamber proximal to the first end. Thus, a passage of blood through the hydrophobic filter is stopped and therefore leakage of blood is prevented.

In an embodiment, the catheter having the rigid conical guide member abutting a disc in the catheter hub provides alignment of the needle inside the catheter hub and thus any injuries due to misalignment of the needle inside the vein is prevented.

In an embodiment, the catheter having the disc made of silicone, provides a self-sealing feature which will prevent the flow of fluid when the needle is withdrawn from the catheter hub.

While aspects of the present invention have been particularly shown and described with reference to the embodiments above, it will be understood by those skilled in the art that various additional embodiments may be contemplated by modification of the disclosed device without departing from the scope of what is disclosed. Such embodiments should be understood to fall within the scope of the present invention as determined based upon claims and any equivalents thereof

### Intravenous Catheter

### LIST OF REFERRAL NUMERALS

100: Catheter or intravenous catheter
102: Catheter tube
104: Proximal end (of catheter tube)
106: Distal end (of catheter tube)
108: Bore (of catheter tube)
110: Needle
112: Catheter hub
112-A: Catheter hub
112-B: Catheter hub
112-C: Catheter hub
114: Inner chamber
116: Distal end (of catheter hub)
118: First fluid pathway
120: Cylindrical passage
122: Projection
122-A: Projection (of tubular sleeve 150-A)
124: Proximal end (of catheter hub)
126: Outer port
128: Outer surface (of catheter hub)
130: Second fluid pathway
132: Dispensing cap
134: Hinge
136: Needle hub
138: Proximal end (of needle)
140: Tip (of needle)
142: Distal end (of needle)
144: Enlarged dimension feature
146: Needle cover
146-A: Needle cover
148: Recess
148-A: Recess
150: Tubular sleeve
150-A: Tubular sleeve
152: Slit
152-A: Slit (of the tubular sleeve 150-A)
154: Bore (of tubular sleeve)
154-A: Bore (of tubular sleeve 150-A)
156: Casing
158: Thumb grip
160: Outer surface (of tubular sleeve)
160-A: Outer surface (of tubular sleeve 150-A)
162: Hooks
164: Slots
166: Flash back chamber
167: Hydrophobic filter
168: Extended portion (of needle hub)
170: Threaded cap
172: Assembly
174: Inner portion
176: Distal end (of tubular sleeve)
176-A: Distal end (of tubular sleeve 150-A)
178: Assembly
180: Thumb portion
182: Outer surface
184: Assembly
186: Thumb portion
188: Outer surface
190: Inner surface
192: Dotted projection
192-A: Dotted projection (of tubular sleeve 150-A)
194: Assembly
195: Inner surface (of catheter hub 112-C)
196: Inner profile
198: Catheter hub
200: Catheter or intravenous catheter
202: Inner chamber (of catheter hub 198)
204: Rigid conical guide member
206: Disc
208: Outer port (of catheter hub 198)
210: Outer surface (of catheter hub 198)
212: Second fluid pathway (of catheter hub 198)
214: Elongated tube
216: Flexible tube
218: Inner chamber (of elongated tube)
220: First end (of elongated tube)
222: Second end (of elongated tube)
224: Through hole
226: First fluid path (of catheter hub 198)
228: Distal end (of the catheter hub 198)
230: Proximal end (of the catheter hub 198)
A-A′: Axis
B-B′: Axis
C-C′: Axis
D-D′: Axis
F-F′: Axis
E1: Enlarged view portion
E2: Enlarged view portion
E3: Enlarged view portion
G: Enlarged view portion

1. An intravenous catheter, comprising:
a flexible plastic catheter tube having a proximal end and a distal end;
a catheter hub having an inner chamber in fluid connection with the catheter tube forming a first fluid pathway, the catheter hub having a distal end connected to the proximal end of the catheter tube having a bore; and
a needle having a proximal end, a tip at a distal end and an enlarged dimension feature towards the distal end, the needle adapted to extend along a length of the bore of the catheter tube and the proximal end of the needle being press fitted in a needle hub, the needle hub connected to a flashback chamber through an extended portion of the needle hub, wherein
a needle cover is connected to the catheter hub by at least one projection and at least one recess configuration, and
wherein the needle cover has a tubular sleeve with an axially oriented radially inward extending slit and a bore for receiving the needle such that when the needle is retracted from the needle cover, the needle cover which is in tight fit relationship with the catheter hub disengages the catheter hub.

2. The intravenous catheter as claimed in claim 1, wherein the tubular sleeve comprises the recess on its outer surface configured to engage with the projection in the catheter hub, wherein the recess is a groove or cavity on the outer surface of the tubular sleeve.

3. The intravenous catheter as claimed in claim 1, wherein the tubular sleeve comprises a projection on the outer surface of the tubular sleeve configured to engage with a recess in the catheter hub, wherein the recess is a groove or cavity on an inner surface of the catheter hub.

4. The intravenous catheter as claimed in claim 1, wherein the needle cover having the tubular sleeve with the slit and the bore is adapted to receive the needle such that when the needle is inserted into the needle cover, the extended dimension feature of the needle pushes the slit outwardly for locking of the needle cover with the catheter hub by engagement of the projection with the recess.

5. The intravenous catheter as claimed in claim 1, wherein the projection is a radially extending ring-like structure or a protruding feature designed to mesh with the recess.

6. The intravenous catheter as claimed in claim 1, wherein the catheter hub comprises an outer port abutting on an outer surface forming a second fluid pathway, wherein the second fluid pathway is in fluid communication with the first fluid pathway.

7. The intravenous catheter as claimed in claim 6, wherein the outer port is provided with a dispensing cap in which a hinge of the dispensing cap has a spring action.

8. The intravenous catheter as claimed in claim 7, wherein the dispensing cap and the outer port are connected together by the hinge urging the dispensing cap to freely swing to a fully open or a fully closed position when pushed in respective directions.

9. The intravenous catheter as claimed in claim 1, further comprising an elongated tube connected to the extended portion of the needle hub.

10. The intravenous catheter as claimed in claim 9, wherein the elongated tube comprises an inner chamber with a first end and a second end, such that the second end is configured to be connected to the extended portion of the needle hub.

11. The intravenous catheter as claimed in claim 10, wherein the elongated tube comprises a hydrophobic filter disposed within the inner chamber proximal to the first end.

12. The intravenous catheter as claimed in claim 1, further comprising a rigid conical guide member abutting a disc in the catheter hub, wherein the disc is made of silicone.

13. The intravenous catheter as claimed in claim 1, wherein the needle cover is connected to a casing, the casing being detachably connected to the needle cover via one or more ribs and to the needle hub via one or more hooks inserted into one or more slots of the needle hub.

14. The intravenous catheter as claimed in claim 1, wherein the needle is a hypodermic needle having a bevelled tip.
